(19) United States
(12) Patent Application Publication
(10) Pub. No.: US 2025/0255542 A1
(43) Pub. Date: Aug. 14, 2025

(54) SYSTEMS AND METHODS OF MUSCULOSKELETAL HEALTH AND PERFORMANCE ASSESSMENT

(71) Applicant: Georgia Tech Research Corporation, Atlanta, GA (US)

(72) Inventors: Omer T. Inan, Atlanta, GA (US); Goktug Ozmen, Atlanta, GA (US); Samer Mabrouk, Atlanta, GA (US); Christopher Nichols, Atlanta, GA (US)

(21) Appl. No.: 18/858,130

(22) PCT Filed: Apr. 21, 2023
(86) PCT No.: PCT/US2023/066069

§ 371 Date: Oct. 18, 2024

Related U.S. Application Data
(60) Provisional application No. 63/363,349, filed on Apr. 21, 2022.

(51) Int. Cl. A61B 5/00; A61B 5/053

(52) U.S. Cl. CPC A61B 5/4519; A61B 5/053; A61B 5/6828

(57) ABSTRACT

An exemplary embodiment of the present disclosure provides a method of assessing musculoskeletal health in a user. The method can comprise: measuring a bioimpedance across a portion of a body of a user at a first time: measuring the bioimpedance across the joint of the user at a second time; and determining, based at least in part on the measured bioimpedances at the first and second times, a change in a biomechanical property of the portion of the body of the user between the first time and the second time.

### CROSS-REFERENCE TO RELATED APPLICATIONS

This application claims the benefit of U.S. Provisional Application Ser. No. 63/363,349, filed on 21 Apr. 2022, which is incorporated herein by reference in its entirety as if fully set forth below.

### GOVERNMENT LICENSE RIGHTS

This invention was made with government support under Agreement No. 1R01EB023808, awarded by National Institutes of Health. The government has certain rights in the invention.

### FIELD OF THE DISCLOSURE

The various embodiments of the present disclosure relate generally to systems and methods for assessing musculoskeletal health and performance.

### BACKGROUND

Physical activity is essential to maintain cardiac, musculoskeletal, and mental health. Unfortunately, excessive activity and overuse can also be harmful with the risk of injury outweighing the benefits of exercising. Across all ages and lifestyles, optimizing the intensity, technique and duration of exercise is critical for achieving the best performance while simultaneously avoiding injury. These cases pose the question: “How much exercise is too much?”

Sports medicine and rehabilitation experts have declared muscle fatigue as a critical metric to quantify for muscular load management and injury prevention. Traditionally, technologies for assessing muscle fatigue are confined to benchtop equipment. For example, ultrasound imaging has become the predominant method of assessing morphological and morphometric characteristics of a muscle. Muscle biopsies are also performed to understand the extent of exercise-induced muscle damage. Additionally, motion capture systems are often employed to detect secondary effects of alterations in muscle. However, these methods are cost-prohibitive, restricted to clinical or laboratory settings, and are not available to a broad population. Along with benchtop measures, subjective performance metrics are also used for performance assessment. One of the most popular and widely used performance metrics is Borg's rating of perceived exertion (RPE) scale. Researchers examined the correlation between RPE and heart rate, oxygen uptake and blood lactate concentrations, but there are inconsistent reports about these relations with weak correlations. In a meta-analysis, researchers previously examined the criterion-related validity of RPE in healthy individuals and concluded that the validity coefficients between RPE and physiological criterion variables were not nearly as high as previously thought.

As an alternative to benchtop systems and subjective questionnaires, wearable technologies could offer a more convenient way for muscle fatigue assessment during exercise when it is most needed, and muscle health monitoring during recovery. One of these physiological sensing modalities is muscle oxygen saturation (SmO₂) measurement, which has been proposed to track the aerobic and anaerobic metabolism of localized muscle groups. This optical measurement captures the changes in oxygenated and deoxygenated hemoglobin concentrations in muscle tissue via the resultant changes in optical absorbances. Unfortunately, these systems can only measure relative concentration changes accurately and are unable to provide sufficient information about possible exercise-induced muscle damage beyond the anaerobic threshold.

One of the most direct measures of localized muscle fatigue is surface electromyography (sEMG). sEMG is a non-invasive method to detect, record and interpret the electrical activity of muscle groups at rest and during activity. Conventionally, sEMG is used to estimate muscle forces through the detection of electrical activation of a muscle. Along with muscle activation, sEMG has been used to detect muscle fatigue during dynamic activities. However, sEMG has its own practical limitations for every day, out-of-the-lab use. For instance, most of the current sEMG systems require skin preparation to avoid artifacts and receive reliable signals because most of the existing signal processing methods assume high quality sEMG data. Another notable concern is the poor longitudinal repeatability of sEMG signals, as the performance of sEMG can degrade by 20-30% over time. These practical issues prevent the reliable use of sEMGs in unsupervised settings. In summary, the current state-of-the-art wearable systems for muscle fatigue tracking suffer from either low specificity or poor usability. Therefore, there is a need for a non-invasive, convenient, and robust wearable system for direct muscle fatigue measurement both during exercise and recovery.

### BRIEF SUMMARY

An exemplary embodiment of the present disclosure provides a method of assessing musculoskeletal health in a user. The method can comprise: measuring a bioimpedance across a portion of a body of a user at a first time; measuring the bioimpedance across the joint of the user at a second time; and determining, based at least in part on the measured bioimpedances at the first and second times, a change in a biomechanical property of the portion of the body of the user between the first time and the second time.

In any of the embodiments disclosed herein, measuring the bioimpedance across the portion of the body of the user at the first time and second time can each comprise: measuring the bioimpedance across the portion of the body of the user while applying a first electrical current across the portion of the body of the user at a first frequency; and measuring the bioimpedance across the portion of the body of the user while applying a first electrical current across the portion of the body of the user at a second frequency.

In any of the embodiments disclosed herein, the first frequency can be about 5 kHz and the second frequency can be about 100 kHz.

In any of the embodiments disclosed herein, determining, based at least in part on the measured bioimpedances at the first and second times, a change in a biomechanical property of the portion of the body of the user between the first time and the second time can comprise calculating a muscle fatigue score.

In any of the embodiments disclosed herein, the muscle fatigue score can be based on a difference between a ratio of the bioimpedances at the first and second frequencies at the first time and the second time.

In any of the embodiments disclosed herein, the biomechanical property can be muscle fatigue.

In any of the embodiments disclosed herein, the biomechanical property can be muscle damage.

In any of the embodiments disclosed herein, the biomechanical property can be muscle torque.

In any of the embodiments disclosed herein, the biomechanical property can be muscle recovery.

In any of the embodiments disclosed herein, the method can further comprise instructing the user to limit use of the portion of the body based on the change in the biomechanical property.

In any of the embodiments disclosed herein, the method can further comprise attaching a bioimpedance measurement system to the portion of the body of the user. The bioimpedance measurement system can comprise a first pair of electrodes, a second pair of electrodes, and a controller. The first pair of electrodes can be positioned proximate a first end of the portion of the body of the user. The second pair of electrodes can be positioned proximate a second end of the portion of the body of the user. The controller can be configured to measure a bioimpedance between the first and second pairs of electrodes.

In any of the embodiments disclosed herein, the bioimpedance measurement system can further comprise an inertial measurement unit configured to measure one or more kinematic properties of the portion of the body of the user.

In any of the embodiments disclosed herein, the first pair of electrodes can be positioned on a thigh of a user at a position above a midpoint of the length of the femur.

In any of the embodiments disclosed herein, the second pair of electrodes can be positioned below a knee of the user.

In any of the embodiments disclosed herein, the portion of the body of the user can comprise the user's knee and at least a portion of one or more muscles of the user above and below the knee.

In any of the embodiments disclosed herein, the method may not comprise measuring an acoustic characteristic of the portion of the body of the user.

Another embodiment of the present disclosure provides a system for assessing musculoskeletal health in a user. The system can comprise a first pair of electrodes, a second pair of electrodes, and a controller. The first pair of electrodes can be configured to be positioned proximate a first end of a portion of a body of a user. The second pair of electrodes can be configured to be positioned proximate a second end of the portion of the body of the user. The controller can be configured to: measure a bioimpedance between the first and second pairs of electrodes at a first time; measure a bioimpedance between the first and second pairs of electrodes at a second time; and determine, based at least in part on the measured bioimpedances at the first and second times, a change in a biomechanical property of the portion of the body of the user between the first time and the second time.

In any of the embodiments disclosed herein, the controller can be configured to measure the bioimpedance between the first and second pairs of electrodes at each of the first and second times by: measuring the bioimpedance between the first and second pairs of electrodes while applying a first electrical current to the first and second pairs of electrodes at a first frequency; and measuring the bioimpedance between the first and second pairs of electrodes while applying a first electrical current to the first and second pairs of electrodes at a second frequency.

In any of the embodiments disclosed herein, the controller can be configured to determine, based at least in part on the measured bioimpedances at the first and second times, a change in a biomechanical property of the portion of the body of the user between the first time and the second time, by calculating a muscle fatigue score.

In any of the embodiments disclosed herein, the controller can be further configured to generate an output instructing the user to limit use of the portion of the body of the user based on the change in the biomechanical property.

In any of the embodiments disclosed herein, the system can further comprise an inertial measurement unit configured to measure one or more kinematic properties of the portion of the body of the user.

These and other aspects of the present disclosure are described in the Detailed Description below and the accompanying drawings. Other aspects and features of embodiments will become apparent to those of ordinary skill in the art upon reviewing the following description of specific, exemplary embodiments in concert with the drawings. While features of the present disclosure may be discussed relative to certain embodiments and figures, all embodiments of the present disclosure can include one or more of the features discussed herein. Further, while one or more embodiments may be discussed as having certain advantageous features, one or more of such features may also be used with the various embodiments discussed herein. In similar fashion, while exemplary embodiments may be discussed below as device, system, or method embodiments, it is to be understood that such exemplary embodiments can be implemented in various devices, systems, and methods of the present disclosure.

### BRIEF DESCRIPTION OF THE DRAWINGS

The following detailed description of specific embodiments of the disclosure will be better understood when read in conjunction with the appended drawings. For the purpose of illustrating the disclosure, specific embodiments are shown in the drawings. It should be understood, however, that the disclosure is not limited to the precise arrangements and instrumentalities of the embodiments shown in the drawings.

FIG. 1 provides a flow chart for a method of assessing musculoskeletal health in a user, in accordance with an exemplary embodiment of the present disclosure.

FIGS. 2A-B illustrate a bioimpedance measurement system, in accordance with an exemplary embodiment of the present disclosure. FIG. 2A provides a photograph of the system being worn by a user. FIG. 2B provides a block diagram of the system.

FIG. 3 provides details of the fatigue protocol and representative raw data from a subject in testing/assessing performance of certain embodiments of the present disclosure. The leg fatiguing protocol comprises 15 sets of 20 split leg squats where participants performed the exercise on their dominant legs. Between each set of split leg squats, participants walked on a treadmill at 1.1 m/s for two-minutes. The exercised leg resistances at both 5 kHz and 100 kHz decreased gradually towards the end of the protocol, while these resistances in control leg did not change noticeably. In addition to this slow change in the average resistance value, mid-activity DFBIA followed a periodic waveform during gait with a distinct shape. The wearable DFBIA system captured high quality data both at the beginning and the end of the protocol. To quantify the slow change in 5 kHz and 100 kHz leg resistances, we calculated their ratio for each gait cycle, R_{5 KHz}/R_{100 KHz}.

FIGS. 4A-D provide derivation of fatigue score and its utility in estimation of percent reduction in muscle force mid-activity, in accordance with some embodiments of the present disclosure. FIG. 4A provides charts illustrating that percent reduction in exercised leg muscle force was significantly larger than that of control leg for both weeks, and the percent reduction in exercised leg muscle force in the first week was significantly larger than that of second week. FIG. 4B provides a chart illustrating that exercised leg fatigue score was significantly larger than that of control leg for both weeks. Also, exercised leg fatigue score in the first week was significantly larger than that of second week. FIG. 4C provides a chart illustrating fatigue score. FIG. 4D provides a chart illustrating estimated reduction in muscle force based on fatigue score, which estimated the percent reduction in muscle force with repeated-measures Pearson's r of 0.90 and a RMSE of 3.6% at a population level (* denotes p<0.05).

FIGS. 5A-C illustrate the effect of DOMS observed both in absolute muscle force and at-home DFBIA, in which the R_{5 kHz}/R_{100 KHz} measured at home has a strong linear effect with muscle force. Muscle force measured in the lab (FIG. 5A) and R_{5 KHz}/R_{100 KHz} measured at home (FIG. 5B) decreased significantly two days after the fatigue protocol on both weeks for 11 participants. R_{5 kHz}/R_{100 KHz} (FIG. 5C) had a strong effect on absolute muscle force with effect size β=646 N, p<0.001 at a population level (198 data points) (* denotes p<0.05).

FIGS. 6A-D plot various VAS scores showing that fatigue score estimates delayed pain quantified bay VAS score better than human perception of exertion during exercise. FIG. 6A shows end of exercise Borg RPE estimates the delayed VAS score poorly with repeated-measures Pearson's r=−0.60, MAE=1.71, and FIG. 6B shows Bland-Altman plot for this estimation shows a 95% adjusted LOA at 4.75. FIG. 6C shows fatigue score estimates the delayed VAS score successfully with repeated-measures Pearson's r=0.83, MAE=0.83, and FIG. 6D shows Bland-Altman plot for this estimation shows a 95% adjusted LOA at 2.01.

FIG. 7 provides a computing device that can be used in various embodiments of the present disclosure.

### DETAILED DESCRIPTION

To facilitate an understanding of the principles and features of the present disclosure, various illustrative embodiments are explained below. The components, steps, and materials described hereinafter as making up various elements of the embodiments disclosed herein are intended to be illustrative and not restrictive. Many suitable components, steps, and materials that would perform the same or similar functions as the components, steps, and materials described herein are intended to be embraced within the scope of the disclosure. Such other components, steps, and materials not described herein can include, but are not limited to, similar components or steps that are developed after development of the embodiments disclosed herein.

Embodiments of the present disclosure can assess intramuscular fluid dynamics to address the need for direct muscle fatigue measurement. Intramuscular fluid dynamics can be critical for the function of biochemistry and biomechanics of muscle activity. The overall fluid content of muscle can dynamically change during exercise, while passive muscle force can be correlated to intramuscular fluid volume. Dual-frequency electrical bioimpedance analysis (DFBIA) can provide a viable solution to the gaps of the conventional methods discussed above. DFBIA is a non-invasive measure of a tissue's electrical characteristics, which can dynamically assess muscle fluid dynamics of humans. Conventionally, DFBIA was used to assess body composition by applying a small alternating current to the body and measuring the change in voltage across it. Localized DFBIA can be used to measure local tissue fluid dynamics for the purpose of musculoskeletal health assessment. This is because the temporal characteristics of tissue resistance and reactance can change significantly when compared to healthy tissue. Thus, because localized DFBIA can be used to detect muscle injuries at specific frequencies, multifrequency DFBIA can be used to assess musculoskeletal health. Low frequency BIA can correlate well with soft tissue extracellular fluid dynamics as these frequencies may not breach the capacitive barrier of cell walls, while high frequency electrical currents can penetrate this capacitive barrier and capture information including both intra- and extracellular fluid dynamics.

Some embodiments of the present disclosure make use of a wearable DFBIA to address the need for a non-invasive, convenient, and robust method of measuring muscle force and pain during physically demanding activities and recovery by assessing intramuscular fluid dynamics. The preset disclosure shows that mid-activity changes in leg DFBIA can estimate delayed onset muscle soreness (DOMS), mid-activity leg DFBIA captures salient indicators of muscle tissue damage, and DOMS is the response of the body to repair the same tissue. Further, embodiments of the present disclosure can advise users on when-to-stop- and when-to-return-exercising by estimating their muscle fatigue at a given time in a convenient and quantifiable way.

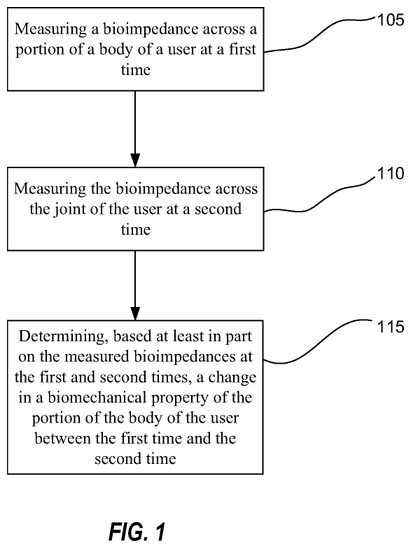

As shown in FIG. 1, an exemplary embodiment of the present disclosure provides a method of assessing musculoskeletal health in a user 115. The method can comprise measuring a bioimpedance across a portion of a body of a user at a first time 105 and measuring the bioimpedance across the joint of the user at a second time 110. Though only two time periods for the measurement are described, as those skilled in the art would appreciate, the disclosure is not so limited. Rather, in some embodiments, many bioimpedance measurements can be taken at various times/time intervals. In some embodiments, bioimpedance measurements can be taken continuously or at predetermined time intervals.

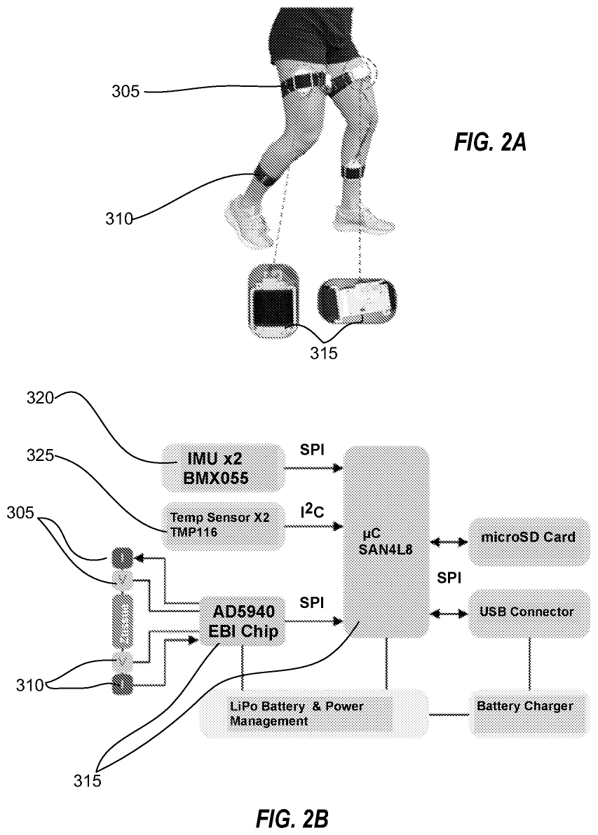

In order to take the bioimpedance measurements, in some embodiments, a bioimpedance measurement system (also referred to herein as a “wearable DFBIA system”) can be used. The bioimpedance measurement system can be placed on the portion of the body of the user. As shown in FIGS. 2A-B, the bioimpedance measurement system 300 can comprise a first pair of electrodes 305, a second pair of electrodes 310, and a controller 315. The first pair of electrodes 105 be positioned proximate a first end of the portion of the body of the user. The second pair of electrodes 310 can be positioned proximate a second end of the portion of the body of the user.

The systems and methods disclosed herein can be used to measure/assess musculoskeletal health/performance of many different portions of the user's body, including, but not limited to, the user's legs, arms, etc. For example, to assess leg performance/health, the portion of the user's body can comprise the user's knee and at least a portion of one or more muscles of the user above and below the knee. In such a case, the first pair of electrodes can be positioned on a thigh of a user (e.g., on muscle(s) on one side of the knee joint), and the second pair of electrodes can be positioned below a knee of the user (e.g., on muscle(s) on the opposite side of the knee joint. In some embodiments, to more accurately assess health/performance, the position of the electrodes can be such that they are not near the knee joint. For example, if the thigh (as related to the femur) of the user can be separated into thirds, the first pair of electrodes can be positioned on either the top or middle third of the thigh. Similarly, the thigh (as related to the femur) of the user can be separated into halves, the first pair of electrodes can be positioned on the top half of the thigh (i.e., above a midpoint of the length of the femur). In some embodiments, the second pair of electrodes can be placed at corresponding positions on the calf (as related to the tibia/fibula) of the user. If arm performance/health is to be assessed, related positions on the arms (e.g., humorous and radius/ulna) can be used.

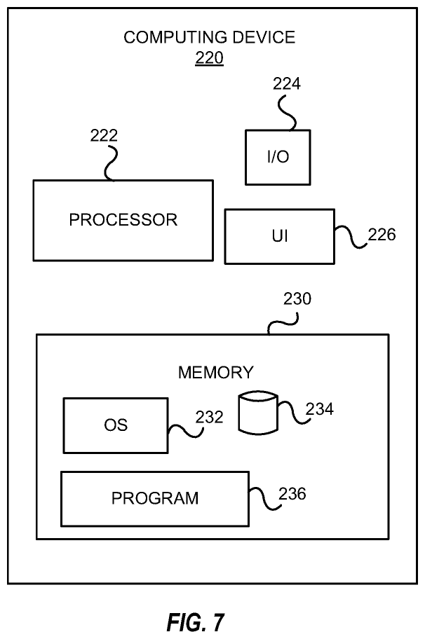

The bioimpedance measurement system can further comprise a controller 315 that can be configured to measure a bioimpedance between the first 305 and second 310 pairs of electrodes. Discussion of exemplary bioimpedance measurements are provided in the Examples below. The controller can be many controllers known in the art. The controller can comprise one or more microcontrollers, CPUs, other computing devices, or combinations thereof. In some embodiments, a portion of the controller can be worn by the user and another portion of the controller can be a remote computing device. In an exemplary embodiment, the controller can be implemented with the computing device 200 shown in FIG. 7 (described below), or one or more components thereof.

In any of the embodiments disclosed herein, the bioimpedance measurement system can further comprise an inertial measurement unit (IMU) 320 configured to measure one or more kinematic properties of the portion of the body of the user, such as acceleration, knee angle, rotation, and the like.

Additionally, in some embodiments, the bioimpedance measurement system 300 can further comprise a temperature sensor 325 configured to measure temperature of the portion of the body of the user.

Data/measurements collected by the temperature sensor and IMU can also be received and processed by the controller 315.

As discussed herein, certain embodiments measure bioimpedance at multiple frequencies. For example, in some embodiments, measuring the bioimpedance across the portion of the body of the user at the first time and second time 105, 110 can each comprise: measuring the bioimpedance across the portion of the body of the user while applying a first electrical current across the portion of the body of the user at a first frequency; and measuring the bioimpedance across the portion of the body of the user while applying a first electrical current across the portion of the body of the user at a second frequency. Additionally, in some embodiments, more than two frequencies can be used for the bioimpedance measurements. The first and second frequencies can be many different frequencies. In some embodiments, the first frequency can be about 5 kHz, and the second frequency can be about 100 kHz, though the disclosure is not so limited.

The method can further comprise determining, based at least in part on the measured bioimpedances at the first and second times, a change in a biomechanical property of the portion of the body of the user between the first time and the second time 115. The biomechanical property can be many different biomechanical properties, including, but not limited to, muscle fatigue, muscle damage, muscle torque (e.g., maximum torque, average torque, etc.), muscle recovery, and the like.

In some embodiments, determining, based at least in part on the measured bioimpedances at the first and second times, a change in a biomechanical property of the portion of the body of the user between the first time and the second time 115 can comprise calculating a muscle fatigue score. As discussed in the Examples below, the muscle fatigue score can be based on a difference between a ratio of the bioimpedances at the first and second frequencies at the first time and the second time.

As discussed herein, the change in the biomechanical property can be an indicator of the performance/health of the muscle/portion of the user's body. Thus, in some embodiments, the method can further comprise generating an output indicative of the change in the biomechanical property. For example, the method can comprise instructing the user to limit use of the portion of the body based on the change in the biomechanical property. This can be accomplished many ways, as would be appreciated by those skilled in the art. For example, a warning light, audible alarm, or similar indication can be generated. An indication could also alert the user that it is safe to resume use of the portion of the body (e.g., after sufficient muscle recovery).

FIG. 7 illustrates an exemplary computing device that can be used to implement the methods (or one or more steps of the methods) disclosed herein. As will be appreciated by one of skill in the art, the computing device 220 can be configured to implement all or some of the features described in relation to the methods 1000 1100. As shown, the computing device 220 may include a processor 222, an input/output (“I/O”) device 224, a memory 230 containing an operating system (“OS”) 232 and a program 236. In certain example implementations, the computing device 220 may be a single server or may be configured as a distributed computer system including multiple servers or computers that interoperate to perform one or more of the processes and functionalities associated with the disclosed embodiments. In some embodiments, computing device 220 may be one or more servers from a serverless or scaling server system. In some embodiments, the computing device 220 may further include a peripheral interface, a transceiver, a mobile network interface in communication with the processor 222, a bus configured to facilitate communication between the various components of the computing device 220, and a power source configured to power one or more components of the computing device 220.

A peripheral interface, for example, may include the hardware, firmware and/or software that enable(s) communication with various peripheral devices, such as media drives (e.g., magnetic disk, solid state, or optical disk drives), other processing devices, or any other input source used in connection with the disclosed technology. In some embodiments, a peripheral interface may include a serial port, a parallel port, a general-purpose input and output (GPIO) port, a game port, a universal serial bus (USB), a micro-USB port, a high definition multimedia interface (HDMI) port, a video port, an audio port, a Bluetooth™ port, a near-field communication (NFC) port, another like communication interface, or any combination thereof.

In some embodiments, a transceiver may be configured to communicate with compatible devices and ID tags when they are within a predetermined range. A transceiver may be compatible with one or more of: radio-frequency identification (RFID), near-field communication (NFC), Bluetooth™, low-energy Bluetooth™ (BLE), WiFi™, ZigBee™, ambient backscatter communications (ABC) protocols or similar technologies.

A mobile network interface may provide access to a cellular network, the Internet, or another wide-area or local area network. In some embodiments, a mobile network interface may include hardware, firmware, and/or software that allow(s) the processor(s) 222 to communicate with other devices via wired or wireless networks, whether local or wide area, private or public, as known in the art. A power source may be configured to provide an appropriate alternating current (AC) or direct current (DC) to power components.

The processor 222 may include one or more of a microprocessor, microcontroller, digital signal processor, co-processor or the like or combinations thereof capable of executing stored instructions and operating upon stored data. The memory 230 may include, in some implementations, one or more suitable types of memory (e.g. such as volatile or non-volatile memory, random access memory (RAM), read only memory (ROM), programmable read-only memory (PROM), erasable programmable read-only memory (EPROM), electrically erasable programmable read-only memory (EEPROM), magnetic disks, optical disks, floppy disks, hard disks, removable cartridges, flash memory, a redundant array of independent disks (RAID), and the like), for storing files including an operating system, application programs (including, for example, a web browser application, a widget or gadget engine, and or other applications, as necessary), executable instructions and data. In one embodiment, the processing techniques described herein may be implemented as a combination of executable instructions and data stored within the memory 230.

The processor 222 may be one or more known processing devices, such as, but not limited to, a microprocessor from the Pentium™ family manufactured by Intel™ or the Turion™ family manufactured by AMD™. The processor 222 may constitute a single core or multiple core processor that executes parallel processes simultaneously. For example, the processor 222 may be a single core processor that is configured with virtual processing technologies. In certain embodiments, the processor 222 may use logical processors to simultaneously execute and control multiple processes. The processor 222 may implement virtual machine technologies, or other similar known technologies to provide the ability to execute, control, run, manipulate, store, etc. multiple software processes, applications, programs, etc. The processor 222 may also comprise multiple processors, each of which is configured to implement one or more features/steps of the disclosed technology. One of ordinary skill in the art would understand that other types of processor arrangements could be implemented that provide for the capabilities disclosed herein.

In accordance with certain example implementations of the disclosed technology, the computing device 220 may include one or more storage devices configured to store information used by the processor 222 (or other components) to perform certain functions related to the disclosed embodiments. In one example, the computing device 220 may include the memory 230 that includes instructions to enable the processor 222 to execute one or more applications, such as server applications, network communication processes, and any other type of application or software known to be available on computer systems. Alternatively, the instructions, application programs, etc. may be stored in an external storage or available from a memory over a network. The one or more storage devices may be a volatile or non-volatile, magnetic, semiconductor, tape, optical, removable, non-removable, or other type of storage device or tangible computer-readable medium.

In one embodiment, the computing device 220 may include a memory 230 that includes instructions that, when executed by the processor 222, perform one or more processes consistent with the functionalities disclosed herein. Methods, systems, and articles of manufacture consistent with disclosed embodiments are not limited to separate programs or computers configured to perform dedicated tasks. For example, the computing device 220 may include the memory 230 that may include one or more programs 236 to perform one or more functions of the disclosed embodiments.

The processor 222 may execute one or more programs located remotely from the computing device 220. For example, the computing device 220 may access one or more remote programs that, when executed, perform functions related to disclosed embodiments.

The memory 230 may include one or more memory devices that store data and instructions used to perform one or more features of the disclosed embodiments. The memory 230 may also include any combination of one or more databases controlled by memory controller devices (e.g., server(s), etc.) or software, such as document management systems, Microsoft™ SQL databases, SharePoint™ databases, Oracle™ databases, Sybase™ databases, or other relational or non-relational databases. The memory 230 may include software components that, when executed by the processor 222, perform one or more processes consistent with the disclosed embodiments. In some examples, the memory 230 may include a database 234 configured to store various data described herein. For example, the database 234 can be configured to store the software repository 102 or data generated by the repository intent model 104 such as synopses of the computer instructions stored in the software repository 102, inputs received from a user (e.g., responses to questions or edits made to synopses), or other data that can be used to train the repository intent model 104.

The computing device 220 may also be communicatively connected to one or more memory devices (e.g., databases) locally or through a network. The remote memory devices may be configured to store information and may be accessed and/or managed by the computing device 220. By way of example, the remote memory devices may be document management systems, Microsoft™ SQL database, SharePoint™ databases, Oracle™ databases, Sybase™ databases, or other relational or non-relational databases. Systems and methods consistent with disclosed embodiments, however, are not limited to separate databases or even to the use of a database.

The computing device 220 may also include one or more I/O devices 224 that may comprise one or more user interfaces 226 for receiving signals or input from devices and providing signals or output to one or more devices that allow data to be received and/or transmitted by the computing device 220. For example, the computing device 220 may include interface components, which may provide interfaces to one or more input devices, such as one or more keyboards, mouse devices, touch screens, track pads, trackballs, scroll wheels, digital cameras, microphones, sensors, and the like, that enable the computing device 220 to receive data from a user.

In example embodiments of the disclosed technology, the computing device 220 may include any number of hardware and/or software applications that are executed to facilitate any of the operations. The one or more I/O interfaces may be utilized to receive or collect data and/or user instructions from a wide variety of input devices. Received data may be processed by one or more computer processors as desired in various implementations of the disclosed technology and/or stored in one or more memory devices.

While the computing device 220 has been described as one form for implementing the techniques described herein, other, functionally equivalent, techniques may be employed. For example, some or all of the functionality implemented via executable instructions may also be implemented using firmware and/or hardware devices such as application specific integrated circuits (ASICs), programmable logic arrays, state machines, etc. Furthermore, other implementations of the computing device 220 may include a greater or lesser number of components than those illustrated.

### Examples

The following examples are for explanatory purposes only and should not be construed as limiting the scope of the present disclosure.

To evaluate the performance of certain embodiments of the present disclosure, a system for assessing musculoskeletal health in a user's leg was used during a 13-day partially-supervised protocol. Throughout the study, participants were asked to use the wearable multimodal system to measure their leg DFBIA in at-home unsupervised settings, as well as to participate in two repeated in-lab fatigue protocols and seven in-lab follow-up measurements.

### Participant Recruitment

Eleven healthy adults [n=11, 4 females and 7 males; age=24.6±2.8 years (means±SD); mass=66.6±17.9 kg; height=1.73±0.13 m] participated in the study. The participants reported no previous history of lower limb musculoskeletal injury or disease, and they were asked to limit their strength training to regular activity levels during the 13-day data collection to reduce confounding activity changes in our DFBIA and muscle force measurements.

### Wearable DFBIA System Design

### Electronic Design of Wearable MFBIA System

An exemplary wearable multimodal MFBIA system shown in FIGS. 2A-B. This design includes a MFBIA front-end with an AD5940 integrated circuit (Analog Devices, Cambridge, MA, USA). A serial peripheral interface (SPI) communication was established between a microcontroller (SAM4L8, Microchip Technology Inc., Chandler, AZ, USA) and the AD5940 chip, and the firmware was programmed to record dual frequency (5 and 100 kHz) MFBIA at a 32 Hz sampling rate. 5 and 100 kHz electrical bioimpedance measurements are shown to be related with extra- and intracellular water content, respectively. The microcontroller sampled two IMUs with a three-axis accelerometer, gyroscope, and magnetometer (BMX055, Bosch Sensortec GmbH, Kusterdingen, Germany) at 100 Hz. In addition, two digital temperature sensors (TMP116, Texas Instruments Inc., Dallas, TX) were used to measure skin temperature sampling every 1 second through a digital I²C interface. The overall block diagram of the system is presented in FIG. 2B. The system was powered by a 500 mAh Li-ion battery. Data acquisition was triggered using a mechanical switch, with the sampled data being saved to a SD card. The data were later transferred to a personal computer for post-processing.

For MFBIA, a four-electrode configuration was employed with Ag/AgCl gel electrodes (3M, MN, USA). To ensure consistent placement of the electrodes, anatomical features were palpated to initially measure the length of the thigh and shank/calf from the greater trochanter to the lateral condyle and from the condyle to the lateral malleolus, respectively. Then, MFBIA electrodes were placed on the middle third portion of the thigh and shank. After the first measurement, participants were asked to mark electrode locations on their legs with a marker, thereby, standardizing the MFBIA configuration.

### Mechanical Design of Wearable MFBIA System

The device form factor comprised three primary components: a main housing and two modular sensor housings. All packaging components were 3D printed with white polylactic acid (PLA) to allow for rapid prototyping of the design. The main housing comprises a battery and a mother printed circuit board (PCB). Each sensor housing also contained a daughter PCB. 20 pin tiger-eye connectors (Samtec, IN, USA) were used as a strong yet low-profile cable connection between the main and sensor housings. A 1 mm thick thermally conductive foam (3M, MN, USA) was placed between the digital temperature sensor—located on the bottom of the daughter PCB—and a hole at the base of the sensor housing. This helped seal of the base of the sensor housings, enabling accurate temperature measurement without allowing sweat and condensation to infiltrate the system. As sweat and movement can degrade the adhesion of gel electrodes over time, elastic Velcro straps with a silicone rubber serpentine pattern were used to secure the main and sensor housings to the participants' legs, thus minimizing the chance of device slippage during dynamic activity.

### Experimental Procedure and Study Design

A 13-day protocol was designed to evaluate performance of the systems and methods for assessing musculoskeletal health disclosed herein. Participants attended an initial in-lab muscle force measurement on Day 1. On Day 3, they were asked to participate in a fatigue protocol while obtaining continuous leg DFBIA and intermittent muscle force measurements. This fatigue protocol was followed by three follow-up muscle force measurements on Days 4, 5, and 6. Then, on Day 10, participants attended a second fatigue protocol similar to the first, followed by three follow-up muscle force measurements on Days 11, 12, and 13. Throughout these 13-days, participants used a wearable system for assessing musculoskeletal health at home without any supervision from the study team to measure their leg DFBIA every morning.

### In-Lab Measurement on Day 1

The first in-lab baseline measurement comprised taking muscle force measurements for all participants using a dynamometer (Biodex System 3 Pro, Biodex Medical Systems Inc., NY, USA). All settings of the dynamometer (the height, left/right and back/front locations of the seat as well as the handle length—the distance between the rotation axis of dynamometer and the harness on which participants pushed during leg flexion) were calibrated in this first session such that the knee of a participant aligned with the rotation axis of the dynamometer. These participant specific settings were then maintained for the remainder of each participant's protocol to ensure measurement consistency. We measured leg extension maximum voluntary contraction (MVC) torques of all participants at a knee angle of 30°. Three muscle force measurements were recorded from each leg, with one-minute rest period in-between each measurement. The average MVC torque of the three measurements were noted as the participant's MVC torque for this session, and it was converted to a force by dividing the torque measurement by handle length of dynamometer. Thigh and waist straps were used during measurements to minimize confounding participant movements and isolate muscle engagement to the leg as much as possible. Participants were verbally encouraged during collection to elicit the maximum torque possible.

### In-Lab Fatigue Protocol

All participants attended two fatigue protocols scheduled one week apart (Days 3 and 10). During this fatigue protocol, baseline muscle force measurements were first taken from both legs of a participant three times each. Two wearable DFBIA systems were then placed on the participants' legs-one device per respective leg—to enable simultaneous DFBIA of both the exercised and control legs. This allowed us to continuously observe the predicted muscle fatigue asymmetry being induced between both legs throughout the protocol.

After donning the wearable DFBIA systems, participants first walked on a treadmill (Bertec, Columbus, OH, USA) at 1.1 m/s for five minutes. The choice of 1.1 m/s was heuristic to help participants walk comfortably while preventing them to start recovering from fatigue. Subsequently, participants did their first set of 20 split leg squats. Participants were asked to squat with their dominant leg while their other leg was supported by a stationary bench. Participants were encouraged to use their full comfortable range of motion to maximize muscle engagement. However, we did not enforce any pace or range of motion as each participant had different baseline levels of athleticism and flexibility. The DFBIA data recorded during this fatigue protocol if referred to as the mid-activity DFBIA.

After the first set of split squats, participants walked for two minutes on a treadmill at 1.1 m/s speed. This alternation between repetitions of split leg squats and two-minutes walking at 1.1 m/s was then repeated for 15 sets total. Following the fifth and tenth set of squats, participants' muscle force from their exercised leg once was measured using the dynamometer. These intermediate muscle force measurements were taken in two minutes including dynamometer set up and participant preparation to be consistent with a two-minute time between consecutive sets of split leg squats. Participants were verbally informed that they could ask for more recovery time or could stop a set of split leg squats short of 20 repetitions if they were struggling excessively. After the completion of the 15th set, the muscle force of both legs of the participants was measured three times. Additionally, after each set of split leg squats, participants were asked to provide a Borg RPE score from 6 to 20, with 6 representing no exertion at all and 20 being maximal exertion. Because a goal was to quantify the perceived exertion by the participants during the fatiguing exercise, split leg squat, Borg RPE scores were obtained after each set of split leg squats. The walking sessions were mainly used to prepare the participants for the next set of split leg squats while not allowing them to recover from fatigue by resting between sets.

Follow-Up In-Lab Measurements after Fatigue Protocol

For the three days following each fatigue protocol, participants were asked to come to the lab for muscle force measurements once per day (Days 4, 5, 6, 11, 12, 13) as literature reports the strongest DOMS effects in the 72 hours following a fatigue exercise. At each visit, muscle force measurements were taken as described above. Of note, muscle force measurements from each participant were taken at approximately the same time of the day for all in-lab measurements to limit confounding factors.

### At-Home Protocol

To assess the utility of wearable DFBIA, each participant was provided a wearable DFBIA system to independently use for at-home data collection. The participants were asked to don the wearable system and measure their leg DFBIA immediately after waking up before excessive walking or movement as our pilot data showed a substantial decrease in measured leg impedances within the first hour of movement after awakening. We attributed this decrease to the rush of blood/fluid to the legs when participants began moving after sleeping in a supine position. We anticipated that the amount of movement and resultant change in leg DFBIA could be different for different participants based on their mobility; therefore, asking participants to use the DFBIA system immediately in the morning helped standardize these unsupervised measurements. Participants measured the DFBIA of both of their legs for one-minute each in a sitting position and were asked to use fresh electrodes for each leg. In addition to DFBIA, participants were asked to provide a visual analog scale (VAS) score for their leg pain from 0 to 10, with 0 representing no pain and 10 indicating very high pain. VAS scores were noted by participants and shared with the study team at the end of the 13-day protocol.

### Summary of the Different Data Types

To summarize the discussion above, an overview of the recorded data, their units, and when these were recorded is presented. The data can be categorized in two paired sets of data corresponding to the time they were collected:
Set 1—Mid-Activity Data
   In-lab mid-activity DFBIA [arbitrary unit (a.u.)] (day 3 and 10)
   In-lab mid-activity muscle force [N] (day 3 and 10)
Borg RPE [a.u.] (day 3 and 10)
Set 2—Follow-up data
   In-lab muscle force [N] (day 1 (baseline), 4, 5, 6, 11, 12 and 13)
   VAS [a.u.] (day 4, 5, 6, 11, 12 and 13)
   At-home DFBIA [a.u.] (day 1 (baseline), 4, 5, 6, 11, 12 and 13)
      Set 1—Mid-activity data
      Set 1—Mid-activity data
      Set 1—Mid-activity data

### Data Analysis

### Processing of In-Lab Fatigue Protocol DFBIA Data

The measured leg DFBIA during the fatigue protocol was converted into real tissue impedances using a calibration scheme. From the DFBIA data, two time series data were obtained for low and high frequency resistances throughout the duration of the fatigue protocol. IMU data was processed to estimate the knee angle during walking. The algorithm used to estimate knee angle first estimates the axis of rotation of the knee and then estimates the knee angle. To avoid the effect of sensor drift on the estimation of knee axis of rotation, the data were divided into 10-second portions and knee axis of rotation, thereby knee angle was estimated for each portion separately. Note that knee angle was only used as context to better understand the DFBIA waveform. Regarding the in lab in-lab fatigue protocol wearable data, the DFBIA data recorded during treadmill walking were considered.

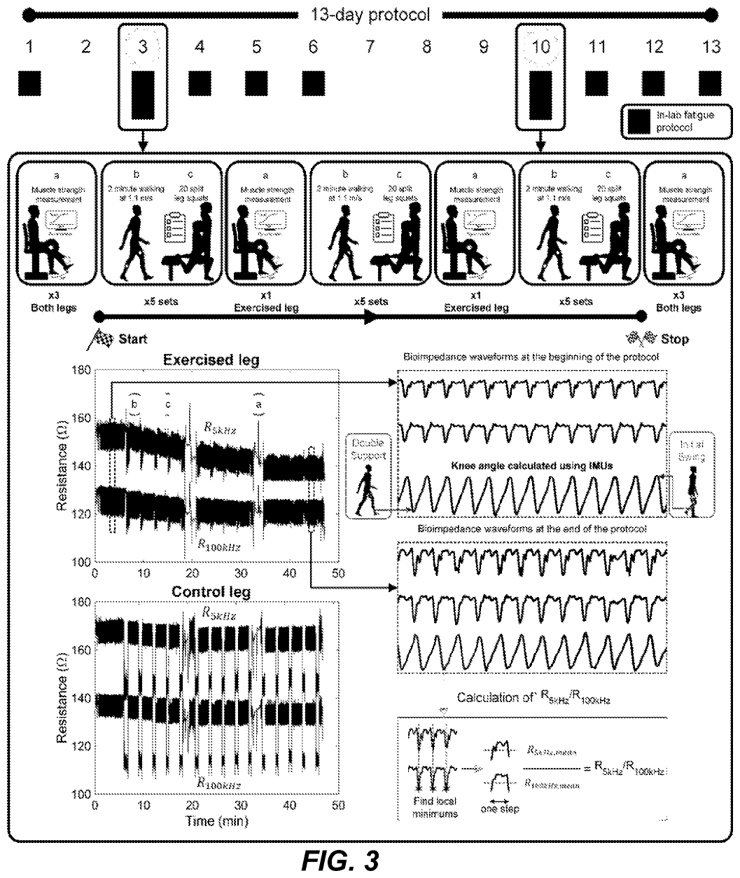

Upon the visual inspection of the DFBIA and knee angle data, it was understood that the DFBIA waveforms are periodic at every gait cycle as presented in FIG. 3. Based on this observation, a simple and interpretable signal processing pipeline was implemented and the walking DFBIA data was segmented into gait cycles by finding the local minimums of R_{100 kHz}. After segmentation, the average of R_{5 kHz} and R_{100 kHz} was calculated within each gait cycle. Here, the average values of R_{5 kHz} and R_{100 kHz} were used so that the analysis would not be affected by behavioral perturbations in gait during the protocol. Within-gait cycle changes are correlated with the changes in vertical ground reaction force. Thereby, such short-term changes in DFBIA might be affected by behavioral factors. The choice of average value instead of other statistical metrics, such as standard deviation, minimum, maximum, provides robustness against these behavioral factors and it is believed to capture physiological changes in soft tissue. From these averages, their ratio was obtained and called as R_{5 kHz}/R_{100 kHz}. That is, each gait cycle was assigned with a R_{5 kHz}/R_{100 KHz}. Because R_{5 kHz} and R_{100 kHz} are correlates of extracellular and overall fluid content of soft tissue, respectively, this ratio quantifies the relative change in extracellular fluid content relative to overall fluid content of soft tissue.

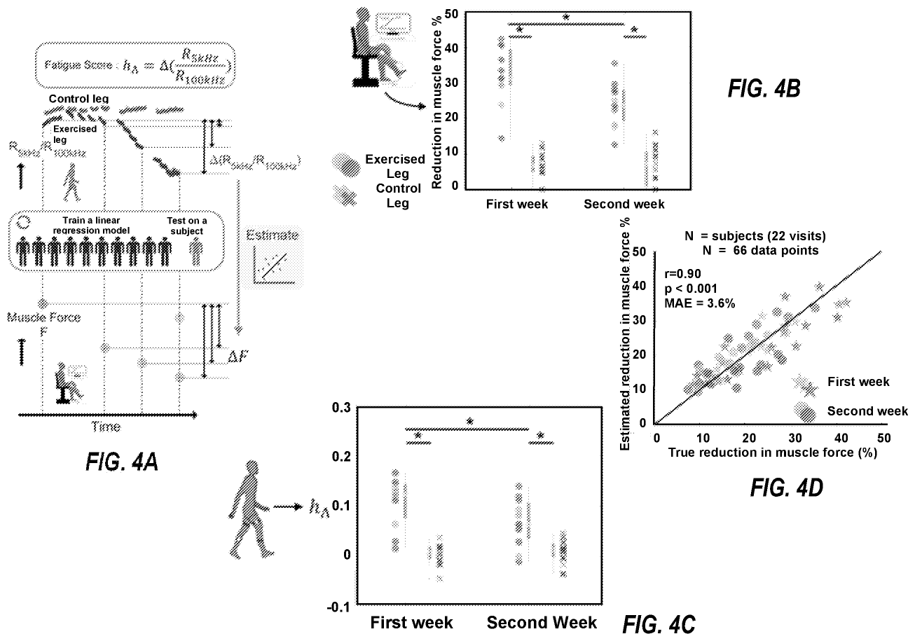

Using the DFBIA ratio of each gait cycle over the course of the fatigue protocol, a time series R_{5 kHz}/R_{100 KHz} was obtained for each leg of a participant. From R_{5 kHz}/R_{100 kHz}, a score, ha, was defined, which is the decrease in R_{5 kHz}/R_{100 kHz} (e.g., at a second/later time period) compared to R_{5 kHz}/R_{100 KHz} of the first gait cycle (e.g., at a first/earlier time period) of a given protocol, A (R_{5 kHz}/R_{100 kHz}) as shown in FIG. 4A. The decrease in R_{5 KHz}/R_{100 KHz} ratio is hypothesized to quantify the relative shift of fluid from intra- to extracellular space. As prior literature suggests the shift of fluid from intra- to extracellular space with fatigue, A (R_{5 KHz}/R_{100 kHz}) can be referred to as a fatigue score.

Processing of at-Home DFBIA Data

The last 30 seconds of each approximately minute-long at-home DFBIA recordings were analyzed to mitigate motion artifacts and electrode wetting time. Using the extracted 30 seconds of data, the average of R_{5 kHz} and R_{100 kHz} and then R_{5 kHz}/R_{100 kHz} were calculated for each participant measurement. Finally, one R_{5 kHz}/R_{100 kHz} value was assigned to each leg for every at-home measurement day.

### Estimation of Mid-Activity Muscle Force Reduction Using Mid-Activity DFBIA

The exercised leg h and Borg RPE score were used to estimate the reduction in exercised leg muscle force during exercise. For this purpose, the average ha of the last walking session and the Borg RPE scores of the split leg squat sets immediately preceding each muscle force measurement were used to estimate the percent reduction in muscle force. The percent reduction in muscle force was then calculated with respect to the baseline muscle force measurement taken at the beginning of fatigue protocol. We acquired three data points for each fatigue protocol per participant, totaling to 66 data points for this estimation.

Once six data points per participant were obtained, two simple linear regression models, one using ha and the other using Borg RPE score as estimators of the percent reduction in muscle force were trained. The repeated-measures Pearson's correlation between true and estimated percent reduction in muscle force and MAE were calculated and reported.

Association Between the Absolute Muscle Force and at-Home DFBIA

Mixed modelling regression with a random intercept per participant was used to investigate the association between the absolute muscle forces of participants and at-home R_{5 kHz}/R_{100 kHz} measurements. The muscle force was calculated by dividing the measured muscle torque by the handle length of the dynamometer for each participant. In total, 198 data points from 11 participants including both exercised and control legs were obtained. In addition, participant specific correlation values were calculated.

### Estimation of Visual Analog Scale (VAS) Score Using Mid-Activity DFBIA

The exercised leg ha and Borg RPE scores were used to estimate the VAS score two days after the fatigue protocol. For this purpose, the average ha of the last walking and Borg RPE score of the last squats exercise at the end of fatigue protocol were used to estimate VAS score. 22 data points from 11 participants were obtained for this estimation.

To estimate VAS scores, two linear regression models were trained through LOPO-CV, one using ha and the other using Borg RPE scores as estimators. The repeated-measures Pearson's correlation coefficient and MAE were calculated for both estimations.

### Statistics

All statistical analyses besides mixed modelling were performed in MATLAB (MathWorks Inc., CA, USA). The repeated-measures Pearson's correlation values were calculated between the true and estimated percent reduction in muscle force, and between the at-home R_{5 kHz}/R_{100 KHz} ratio and absolute muscle force. For these correlations, the null hypothesis that the correlation between two variables in the sample data occurred by chance (α=0.05) was tested. Correlations were computed using an established repeated measures approach to account for multiple measurements per participant. We reported means and standard errors of the mean (SEMs) for absolute muscle force, percent reduction in muscle force, R_{5 kHz}/R_{100 KHz}, and ha. A Friedman's ANOVA test revealed a significant difference between absolute muscle force and R_{5 kHz}/R_{100 kHz} of 11 participants on 9 different days (α=0.05). As a post-hoc test, a Wilcoxon signed rank test was used for pairwise comparisons (α=0.05). These non-parametric tests were chosen because the data had inhomogeneous variances and was not normally distributed.

To investigate the linear association between R_{5 kHz}/R_{100 KHz} and absolute muscle force, mixed models were used. This enabled the estimate of the overall rate of change (B) between R_{5 KHz}/R_{100 kHz} and absolute muscle force while accounting for repeated measures via a random intercept per participant. Mixed models were fit using the lme4 package, where p-values were obtained using Satterthwaite's degrees of freedom method.

### Results

### Leg DFBIA Decreases Gradually Towards the End of the Fatiguing Protocol

Representative mid-activity leg DFBIA data from a participant are presented in FIG. 3. Participants were asked to perform split leg squats to induce asymmetric leg fatigue: participants thereby experienced fatigue in the leg performing the squat, while the contralateral leg experienced minimal fatigue. This strategy allowed for the investigation of both inter- and intra-participant variability. For the remainder of this disclosure, the leg on which a participant placed their weight during the split leg squats is referred to as the exercised leg, and the contralateral leg is referred to as the control leg.

For all participants, the 5 and 100 kHz resistances (R_{5 kHz} and R_{100 kHz}, respectively) of the exercised leg exhibited a decreasing trend throughout the protocol, whereas no consistent directional change was noted in the corresponding resistance values from the control leg. In addition to these gradual changes in baseline leg resistances, mid-activity DFBIA data were characterized by distinct and repeated morphologies within each gait cycle. Two snapshots of these waveforms from the beginning and end of the protocol are presented in FIG. 3. Throughout the protocol, the wearable DFBIA system produced reliable leg resistance measurements with distinct DFBIA waveform patterns at characteristic instances of the gait. To quantify the relative change in R_{5 kHz} and R_{100 KHz} baseline values, the DFBIA waveforms were segmented into gait cycles by finding the local minimums of R_{100 KHz}. Then, the averages of R_{5 kHz} and R_{100 kHz} within each gait cycle were calculated and divided to obtain R_{5 kHz}/R_{100 kHz} ratio for a given gait cycle.

### Fatigue Score Estimates Reduced Muscle Force During the Fatiguing Protocol

The changes in R_{5 kHz}/R_{100 kHz} and muscle force between the exercised and control legs as well as between the first and second weeks of all subjects were compared. As shown in FIG. 4A, the decrease in the R_{5 kHz}/R_{100 KHz} ratio in the exercised leg follows a similar pattern to the reduction in the exercised leg muscle force. To better quantify this relation, a fatigue score, ha, was defined, which is the decrease in R_{5 kHz}/R_{100 kHz} at a given time compared to the baseline (e.g., earlier time) R_{5 kHz}/R_{100 kHz}, measured at the beginning of the exercise. A positive ha indicates a decreasing trend in R_{5 KHz}/R_{100 KHz}.

As shown in FIG. 4B, the first week of the protocol revealed a reduction in the exercised leg muscle force that was significantly higher than that of the control leg. In the second week, the reduction in exercised leg muscle force was also significantly higher than that of control leg. The reduction in exercised leg muscle force during first week, however, was significantly higher than that of the second week. We did not observe any significant change in the control leg muscle force between the first and second weeks.

As shown in FIG. 4C, the fatigue score, ha, of the exercised leg during the first week was significantly higher than that of the control. This difference was also present in the second week, with the ha of the exercised leg being significantly higher than that of the control leg. Similar to the trend seen in muscle force reduction, the ha of the exercised leg during first week was significantly higher than that of the second week. No significant change in ha for the control leg between the first and second weeks was observed.

For all participants, the skin temperature of each leg was measured as the average of the temperature recorded on the thigh and shank. There was no significant difference between the change in the skin temperature of the exercised (3.00±0.26° C. (means±SEM)) and control (2.79±0.21° C. (means±SEM)) legs for either week (Wilcoxon signed rank, two-tailed, n=22, P=0.1778).

Notably, the total of two skin-electrode impedances and tissue impedance was always below 2.5 kΩ at 5 kHz throughout all data collections (n=22 visits). The DFBIA integrated circuit operates linearly for high skin-electrode impedances, and the effect of skin-electrode impedance in our measurements was further investigated. Fluctuations of skin-electrode impedance altered the measured 5 kHz resistance on average by only 0.33Ω.

The ha metric was used to estimate the percent reduction in the mid-activity muscle force for the exercised leg as shown in FIG. 4D. For each participant, there are six data points—three data points per exercise day-which correspond to the three muscle force measurements taken during exercise with respect to the baseline measurement taken at the beginning of the protocol. A leave-one-participant-out cross-validation (LOPO-CV) was used to train and test a simple linear regression model across all participants. The ha estimated the percent reduction in muscle force with repeated-measures Pearson's r of 0.90 and mean absolute error (MAE) of 3.6% (repeated measures correlation, N=11 participants, n=66 data points, p<0.001). In FIG. 4E, participant specific correlations are also presented with all subjects having Pearson's r greater than 0.8. Additionally, the same estimation was performed using Borg RPE scores as the estimator and found that the MAE per participant when estimating the reduction in muscle force with the ha was significantly smaller compared to estimating with Borg RPE score (Wilcoxon signed rank, two-tailed, N=11 participants, P=0.0137).

At-Home DFBIA Correlates with Absolute Muscle Force

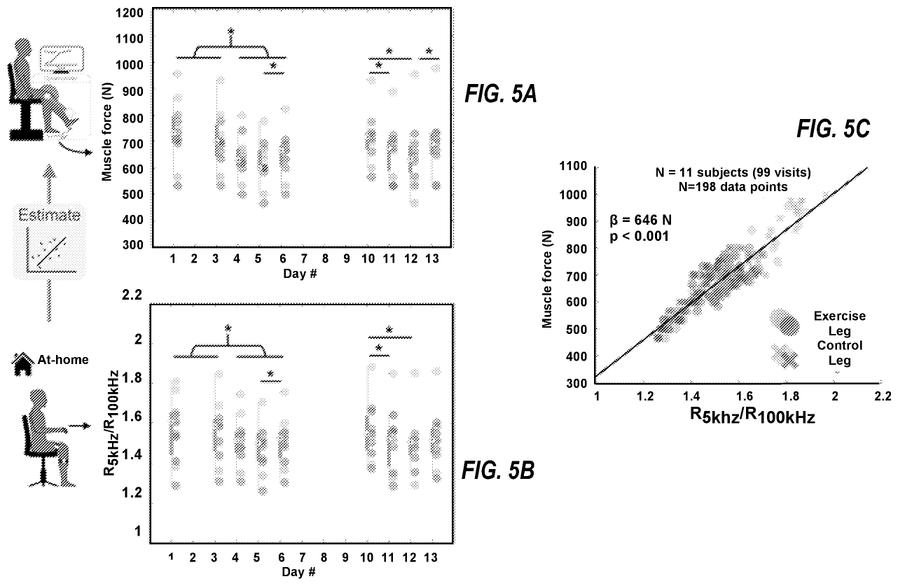

The static R_{5 kHz}/R_{100 KHz}, i.e., at home DFBIA while at rest, and the muscle force of all participants was compared over the course of 13 days as shown in FIG. 5. In FIG. 5A, significant differences were found in muscle force between all days (Friedman's ANOVA, two-tailed, N=90, p<0.001). Then, two-tailed Wilcoxon signed rank test were run as a post-hoc test for pairwise comparisons and several statistically significant differences between muscle forces values at different days were found.

In FIG. 5B, significant differences in R_{5 kHz}/R_{100 KHz} were found between all days (Friedman's ANOVA, two-tailed, N=90, P<0.001). Then, similar to the muscle force measurements, two-tailed Wilcoxon signed rank test were run as a post-hoc test for pairwise comparisons and several statistically significant differences between R_{5 kHz}/R_{100 kHz} values at different days were found.

The R_{5 kHz}/R_{100 kHz} values obtained through at-home DFBIA were strongly associated with the muscle force measurements taken in the lab on the same day as shown in FIG. 5C (N=11 participants, n=198 data points, β=646 N, P<0.001). This association implies that for every 0.1 unit increase in R_{5 kHz}/R_{100 kHz}, there is a 64.6 N increase in absolute muscle force. In FIG. 5D, participant specific Pearson's correlations were presented with all participants showcasing a Pearson's r>0.7.

Mid-Activity Fatigue Score Predicts DOMS Significantly Better than Borg RPE

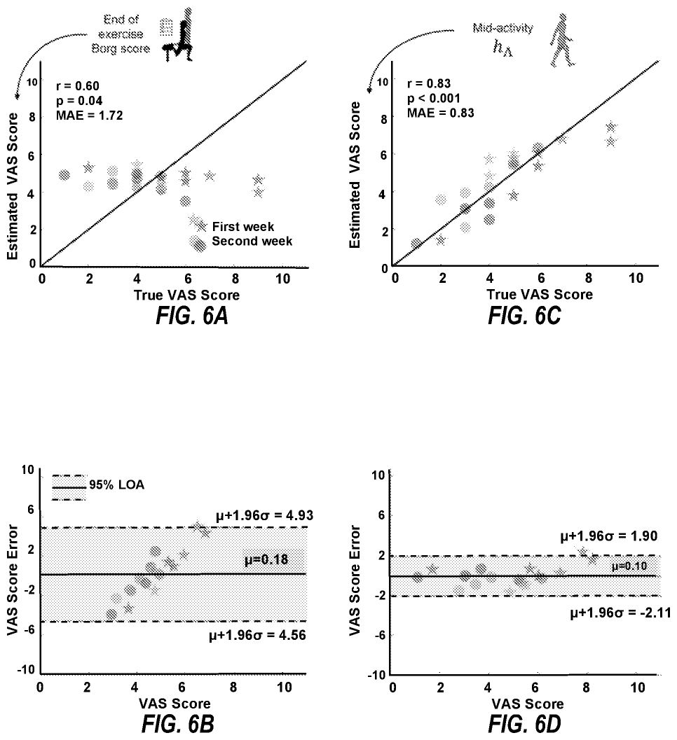

The two estimators from the exercise protocol—the end of exercise fatigue scores, ha and Borg RPE scores-on the exercise days (Days 3 and 10) were used to estimate the leg pain VAS scores for the two days post exercise (Days 5 and 12). The estimation through LOPO CV revealed that ha estimated the VAS scores significantly better than Borg RPE scores. The ha metric successfully estimated the VAS score with a strong correlation (Repeated-measures, N=11 participants, n=22 data points, Pearson's r=0.83, MAE=0.83, p<0.001), whereas the end of exercise Borg RPE score produced poor estimations of the VAS score (Repeated-measures, N=11 participants, n=22 data points, Pearson's r=0.60, MAE=1.72, p=0.04), as shown in FIGS. 6A & 6C. The MAE per participant when estimating the VAS score with the ha was significantly smaller compared to estimating VAS score with Borg RPE score (Wilcoxon signed rank, two-tailed, N=11 participants, P=0.0099). Moreover, the VAS score estimation with ha improved the limit of agreement between the true and estimated VAS score by 58% when compared to the estimation derived from Borg RPE score. The adjusted limit of agreement (LOA) with Borg RPE score and ha around their respective means were 4.75 and 2.01, respectively.

### Discussion

It was demonstrated that (1) mid-activity DFBIA measures can enable estimation of the percent reduction in muscle force for fatiguing exercises, (2) at-home DFBIA values can allow for estimating absolute maximum muscle force, and (3) mid-activity changes in DFBIA can enable prediction of DOMS. This is the first time that wearable DFBIA were shown to track muscle fatigue in dynamic unsupervised settings. Furthermore, the fatigue score, ha, robustly estimates both muscle force reduction during exercise as well as DOMS. These findings demonstrate the effect of intramuscular fluid dynamics on muscle fatigue and have important implications for the utility of a wearable DFBIA system for human performance optimization and injury prevention.

The wearable systems to assess muscle fatigue disclosed herein were user-friendly and convenient. In the study, participants were able to don the device and collect multimodal measurements without researcher assistance. The user interface of the systems included only one on/off switch so that participants could easily collect the measurements with minimal training. Additionally, none of the participants reported any device-related discomfort, nor did any of the devices malfunction during the 22 multi-hour in-lab visits or the 198 unsupervised at-home data collections. This indicates that the electrical and mechanical design of the system were sufficiently robust for at-home use. The convenience, robustness, and low computational requirements of the wearable DFBIA system suggest that it can be readily adapted for use during rehabilitation and exercise by untrained individuals.

While the DFBIA system collected high quality data throughout the study, the findings can be elaborated to better understand why DFBIA is a promising sensing modality to non-invasively assess intramuscular fluid dynamics and thereby muscle fatigue. The findings with the in-lab portion of the study suggest that R_{5 kHz} leg resistance decreases substantially compared to R_{100 KHz} with reduced muscle force. Because low frequency current may only flow through extracellular content while high frequency current can flow in both intra- and extracellular content, this suggests that a post-exercise fluid shift from intracellular to extracellular space occurs, and the overall fluid content in the leg increases.

Based on these physiological reasons why R_{5 kHz} and R_{100 kHz} decrease with muscle fatigue, the decrease in R_{5 KHz}/R_{100 kHz}, ha, was defined to relate these changes to the dynamic changes in muscle force, which can be critically important to optimize human performance and prevent injuries. To derive ha, R_{5 kHz}/R_{100 KHz} which accounts for the body's overall response to exercise through R_{100 KHz}, was first calculated, and the net shift of fluid into extracellular space was simultaneously measured. The decrease in this ratio may indicate localized muscle inflammation, which is expected to follow reduced muscle force with fatigue. This relation can contextualizes to the results presented in FIG. 4D, where the larger the change in R_{5 KHz}/R_{100 KHz}, and therefore ha, the larger the reduction in muscle force. This demonstrates the utility of ha to monitor the muscular load of a person during exercise. While the subjectivity of Borg RPE caused it to perform poorly, ha estimates mid-activity reduction in muscle force unencumbered by user perception, thus offering a more robust way of mid-activity muscle force assessment. Therefore, ha shows potential as a digital biomarker of muscle health to augment human performance by increasing the duration of high-performance activity with reduced risk of injury.

As presented in FIGS. 4B-C, significantly less reduction in exercised leg muscle force and a smaller exercised leg ha was observed in the second week compared to the first week. The smaller reduction in muscle force in the second week is likely explained by muscular adaptation accompanied by an increase in sarcomere count in muscle fibers. Notably, we observed less than 10% control leg muscle force reduction on average, while ha was always less than 0.05 in both weeks. Participants experienced minimal fatigue in their control legs and had a significantly smaller fatigue score in their control legs compared to exercised legs. These observations highlight the ability of mid-activity DFBIA to detect asymmetric leg fatigue and imply that this technology may precent lower limb injuries originated by asymmetric loading.

While measuring mid-activity change in leg muscle force could be beneficial for augmenting human performance and preventing injuries, assessing muscle health and performance during recovery can also be essential to make informed decisions on rehabilitation and training programs. To this end, as demonstrated in FIG. 5C, the measurement of DFBIA obtained at home in the morning was strongly associated with absolute muscle force that could be achieved later in the day. Prior literature has shown that as extra- to intracellular water ratio in the muscle decreases, muscle force increases. In the context of the measurements from the study, this implies that R_{5 kHz}/R_{100 kHz} increases, with muscle force. This observation could be explained by the fact that increased force is accompanied by muscle hypertrophy-which correspondingly increases the amount of total cytoplasm and intracellular water within the muscle.

The high correlation observed between participant specific data for the estimation of absolute muscle force however, may not be explained by muscle hypertrophy, as two exercise sessions during our 13-day protocol would be unlikely to produce any noticeable changes. Rather, the participants experienced DOMS, which is known to induce muscle swelling and reduce muscle force while damaged muscle fibers are being repaired. DOMS has been shown to increase muscle extracellular water content more substantially than intracellular water content. Therefore, with DOMS it is expected that R_{5 kHz}/R_{100 kHz} ratio and muscle force decrease, as presented in FIGS. 5A-B, respectively. These findings imply that individuals could use the wearable DFBIA systems disclosed herein at home every morning to assess their baseline leg muscle force. The globalized and participant specific correlations between DFBIA and absolute muscle force suggest that a wearable DFBIA system can inform individuals about their absolute leg muscle force at any given day. This way, wearable DFBIA could help advise individuals on “when-to-return” exercising or rehabilitation to optimize human performance and reduce injury rate.

While muscle force can be an important factor affecting human performance, the pain perceived by individuals could also alter their performance. Exercise-induced pain due to exercise-induced muscle damage could be quantified by mid-activity DFBIA. Therefore, mid-activity changes in DFBIA can estimate muscle pain related to DOMS and outperform Borg RPE estimations as presented in FIG. 6C. As there are several physiological and mental confounders contributing to one's overall perception of their exertion, Borg RPE scores provided by the participants were highly subjective with low resolution. In contrast, it was shown that he can be an objective physiologically-derived metric of muscle damage and resulting inflammation during exercise. Therefore, in addition to its use to estimate mid-activity reduction in muscle force, ha could also be used to estimate DOMS. This way, individuals could be advised on “when-to-stop” exercising or rehabilitation to minimize DOMS for sustained performance and faster recovery.

It is to be understood that the embodiments and claims disclosed herein are not limited in their application to the details of construction and arrangement of the components set forth in the description and illustrated in the drawings. Rather, the description and the drawings provide examples of the embodiments envisioned. The embodiments and claims disclosed herein are further capable of other embodiments and of being practiced and carried out in various ways. Also, it is to be understood that the phraseology and terminology employed herein are for the purposes of description and should not be regarded as limiting the claims.

Accordingly, those skilled in the art will appreciate that the conception upon which the application and claims are based may be readily utilized as a basis for the design of other structures, methods, and systems for carrying out the several purposes of the embodiments and claims presented in this application. It is important, therefore, that the claims be regarded as including such equivalent constructions.

Furthermore, the purpose of the foregoing Abstract is to enable the United States Patent and Trademark Office and the public generally, and especially including the practitioners in the art who are not familiar with patent and legal terms or phraseology, to determine quickly from a cursory inspection the nature and essence of the technical disclosure of the application. The Abstract is neither intended to define the claims of the application, nor is it intended to be limiting to the scope of the claims in any way.

1. A method of assessing musculoskeletal health comprising:
measuring a bioimpedance across a portion of a body of a user at a first time;
measuring the bioimpedance across the portion of the body of the user at a second time; and
determining, based at least in part on the measured bioimpedances at the first and second times, a change in a biomechanical property of the portion of the body of the user between the first time and the second time.

2. The method of claim 1, wherein measuring the bioimpedances comprises:
measuring the bioimpedance at the first time while applying, with a wearable system, a first electrical current across the portion of the body of the user at a first frequency; and
measuring the bioimpedance at the second time while applying, with the wearable system, a first electrical current across the portion of the body of the user at a second frequency.

3. The method of claim 2, wherein the first frequency is about 5 kHz and the second frequency is about 100 kHz.

4. The method of claim 2, wherein the biomechanical property is selected from a group consisting of pain and muscle fatigue.

5. The method of claim 4, wherein the biomechanical property is muscle fatigue; and
wherein determining comprises calculating a muscle fatigue score based on a difference between a ratio of the bioimpedances at the first and second frequencies at the first time and the second time.

6. The method of claim 1, wherein the biomechanical property is selected from a group consisting of muscle fatigue, muscle damage, muscle torque, and muscle recovery.

7.-9. (canceled)

10. The method of claim 2, wherein:
measuring the bioimpedances comprises measuring the bioimpedances of the portion of the body of the user during dynamic activity of the portion of the body; and
determining uses dual-frequency electrical bioimpedance analyses (DFBIA).

11. The method of claim 10 further comprising:
upon determining mid-activity changes in the portion of the body DFBIA during the dynamic activity, reporting at least one of:
an indication representative of delayed onset muscle soreness (DOMS); or
an indication representative of muscle tissue damage;
wherein the wearable system comprises a bioimpedance measurement system comprising:
a first pair of electrodes positioned proximate a first end of the portion of the body of the user;
a second pair of electrodes positioned proximate a second end of the portion of the body of the user; and
a controller configured to measure a bioimpedance between the first and second pairs of electrodes.

12. The method of claim 11, wherein the bioimpedance measurement system further comprises an inertial measurement unit configured to measure one or more kinematic properties of the portion of the body of the user.

13. The method of claim 11, wherein the first pair of electrodes are positioned on a thigh of a user at a position above a midpoint of the length of the femur; and
wherein the second pair of electrodes are positioned below a knee of the user.

14. (canceled)

15. The method of claim 1, wherein the method does not comprise measuring an acoustic characteristic of the portion of the body of the user.

16. (canceled)

17. A system for assessing musculoskeletal health comprising:
a first pair of electrodes configured to be positioned proximate a first end of a portion of a body of a user;
a second pair of electrodes configured to be positioned proximate a second end of the portion of the body of the user; and
a controller configured to assess musculoskeletal health of the portion of the body of the user according to the method of claim 1;
wherein the controller is further configured to:
measure the bioimpedance between the first and second pairs of electrodes at the first time; and
measure the bioimpedance between the first and second pairs of electrodes at the second time.

18. The system of claim 17, wherein the controller is further configured to measure the bioimpedance between the first and second pairs of electrodes at each of the first and second times by:
measuring the bioimpedance between the first and second pairs of electrodes while applying a first electrical current to the first and second pairs of electrodes at a first frequency; and
measuring the bioimpedance between the first and second pairs of electrodes while applying a first electrical current to the first and second pairs of electrodes at a second frequency.

19. The system of claim 18, wherein the first frequency is about 5 kHz and the second frequency is about 100 kHz.

20. The system of claim 18, wherein the controller is further configured to determine, based at least in part on the measured bioimpedances at the first and second times, a change in a biomechanical property of the portion of the body of the user between the first time and the second time, by calculating a muscle fatigue score.

21. The system of claim 20, wherein the muscle fatigue score is based on a difference between a ratio of the bioimpedances at the first and second frequencies at the first time and the second time.

22. The system of claim 17, wherein the biomechanical property is selected from a group consisting of muscle fatigue, muscle damage, muscle torque, and muscle recovery.

23.-25. (canceled)

26. The system of claim 17, wherein the controller is further configured to generate an output instructing the user to limit use of the portion of the body of the user based on the change in the biomechanical property.

27. The system of claim 17 further comprising an inertial measurement unit configured to measure one or more kinematic properties of the portion of the body of the user.

28. The system of claim 17, wherein at least one of:
the first pair of electrodes are configured to be positioned on a thigh of the user at a position above a midpoint of the length of the femur, and wherein the second pair of electrodes are configured to be positioned below a knee of the user;
the portion of the body of the user comprises the user's knee and at least a portion of one or more muscles of the user above and below the knee; or
the system does not include a sensor for measuring an acoustic characteristic of the portion of the body of the user.

29.-30. (canceled)
